(19) United States
(12) Patent Application Publication
(10) Pub. No.: US 2014/0349878 A1
(43) Pub. Date: Nov. 27, 2014

(54) PACLITAXEL RESPONSE MARKERS FOR CANCER

(71) Applicant: NATIONAL RESEARCH COUNCIL OF CANADA, Ottawa (CA)

(72) Inventors: Edwin Wang, Laval (CA); Jie Li, Montreal (CA); Maureen O'Connor-McCourt, Beaconsfield (CA); Enrico Purisima, Pierrefonds (CA)

(73) Assignee: NATIONAL RESEARCH COUNCIL OF CANADA, Ottawa, ON (CA)

(21) Appl. No.: 14/361,153

(22) PCT Filed: Nov. 27, 2012
(86) PCT No.: PCT/CA2012/001087

§ 371 Date: May 28, 2014

Related U.S. Application Data
(60) Provisional application No. 61/563,929, filed on Nov. 28, 2011.

(51) Int. Cl. C12Q 1/68

(52) U.S. Cl. CPC C12Q 1/6886; C12Q 2600/106; C12Q 2600/136; C12Q 2600/16
USPC 506/9; 506/16

(57) ABSTRACT

Cancer marker sets consisting of particular genes differentially expressed in tumours provide improved accuracy of predicting effectiveness of paclitaxel or paclitaxel-like drug treatment against a cancer. These sets are further useful for screening drug candidates for paclitaxel-like cancer treatment activity. The cancer marker sets may be used in a clinical setting to provide information about the likelihood that a cancer patient would or would not respond to paclitaxel or paclitaxel-like drug treatment.

### CROSS-REFERENCE TO RELATED APPLICATIONS

This application claims the benefit of U.S. Provisional Patent Application Ser. No. 61/563,929 filed Nov. 28, 2011, the entire contents of which is herein incorporated by reference.

### FIELD OF THE INVENTION

The present invention is related to cancer, more particularly to methods and markers for predicting whether paclitaxel would be effective for treating a tumour in a patient, and to methods and markers for screening drug candidates for paclitaxel-like tumour treating activity.

### BACKGROUND OF THE INVENTION

Cancer is the second most common cause of death in the Western world, where the lifetime risk of developing cancer is approximately 40%. The overall annual costs of cancer, measured in direct medical expenses and lost productivity, is increasing at an exponential rate. In 2008 costs were estimated to be $228 billion in the United States alone (La Thangue 2011). In general, one cancer drug is only effective in a small fraction (10-30%) of cancer patients (Sarker 2007). Therefore, predictive biomarker-driven cancer therapy could lead to a reduction in unnecessary treatment (reducing healthcare cost) and adverse effects.

Predictive biomarkers for drug response are sets of genes/proteins whose modulated levels could be used to determine whether a patient would or would not respond to a particular drug. Paclitaxel is a drug that targets a cancer cell's essential cell-cycle processes, and has become a first line drug for treating various cancers, for example breast cancer, ovarian cancer and prostate cancer. However, similar to other cancer drugs, only a small fraction of patients respond to paclitaxel treatment, for example only 20% of ER+ breast cancer patients and 30% of ERN triple negative breast cancer patients respond to paclitaxel. Therefore, it would be useful to have biomarkers to predict whether a patient would respond or not to treatment with paclitaxel. Current efforts have been made to identify such biomarkers; however, prediction rates are in the range of 50-60% (Hatzis 2011), which is still too low to be truly useful.

Recently, an algorithm (Multiple Survival Screening (MSS)) has been developed for identifying high-quality cancer prognostic markers and this algorithm was applied for identifying robust marker sets for breast cancer prognosis (Li 2010; Wang 2010).

There is a need to find new markers and develop new tests which are able to more accurately and robustly predict which patients would respond or not respond to paclitaxel or paclitaxel-like drug treatment.

### SUMMARY OF THE INVENTION

It has now been found that marker sets consisting of particular genes differentially expressed in tumours advantageously provide improved accuracy of predicting effectiveness of paclitaxel or paclitaxel-like drug treatment against a cancer. These sets are further useful for screening drug candidates for paclitaxel-like tumour treatment activity. The marker sets of the present invention may be used in a clinical setting to provide information about the likelihood that a cancer patient would or would not respond to paclitaxel or paclitaxel-like drug treatment.

In one aspect of the present invention, there is provided a method of determining likelihood that a tumour in a patient would be treatable with paclitaxel or a paclitaxel-like drug, the method comprising: obtaining a gene expression list of a sample of the tumour or an extract of the tumour having message RNA therein of the patient; determining a gene expression profile of the sample from the gene expression list for genes of a gene marker set; and, comparing the gene expression profile of the sample to standardized “good” and “bad” profiles of the marker set to determine whether the gene expression profile of the sample predicts that the tumour is treatable or not treatable with paclitaxel or a paclitaxel-like drug, wherein “good” indicates that the tumour is likely treatable with paclitaxel or a paclitaxel-like drug and “bad” indicates that the tumour is not likely treatable with paclitaxel or a paclitaxel-like drug.

In a second aspect of the invention, there is provided a method of screening a chemical compound as a drug candidate with paclitaxel-like tumour-treating activity, the method comprising: determining a gene expression profile for genes of a gene marker set of a tumor sample treated with the chemical compound; and, comparing the gene expression profile of the sample to standardized “good” and “bad” profiles of the marker set to determine whether the gene expression profile of the sample predicts that the chemical compound would have paclitaxel-like tumour-treating activity, wherein “good” indicates that the chemical compound is likely to have paclitaxel-like tumour-treating activity and “bad” indicates that the tumour is not likely to have paclitaxel-like tumour-treating activity.

In methods of the present invention, the gene marker set is one or more of Set 1, Set 2, Set 3, Set 4, Set 5 and Set 6, wherein

### Set 1:

| Gene Name | EntrezGene ID | Full Name of Gene |
|---|---|---|
| HELLS | 3070 | Helicase, lymphoid-specific |
| CDC2 | 983 | Cell division cycle 2, G1 to S and G2 to M |
| PLEKHF1 | 79156 | Pleckstrin homology domain containing, family F (with FYVE domain) |
| | | member 1 |
| IGFBP3 | 3486 | Insulin-like growth factor binding protein 3 |
| CASP3 | 836 | Caspase 3, apoptosis-related cysteine peptidase |
| HRK | 8739 | Harakiri, BCL2 interacting protein (contains only BH3 domain) |
| PCSK6 | 5046 | Proprotein convertase subtilisin/kexin type 6 |
| PLAGL1 | 5325 | Pleiomorphic adenoma gene-like 1 |
| NME5 | 8382 | Non-metastatic cells 5, protein expressed in (nucleoside-diphosphate |
| | | kinase) |
| PROP1 | 5626 | PROP paired-like homeobox 1 |
| NOD2 | 64127 | Nucleotide-binding oligomerization domain containing 2 |
| CD38 | 952 | CD38 molecule |
| ATP7A | 538 | ATPase, Cu++ transporting, alpha polypeptide (Menkes syndrome) |
| INDO | 3620 | Indoleamine-pyrrole 2,3 dioxygenase |
| PIM2 | 11040 | Pim-2 oncogene |
| ECT2 | 1894 | Epithelial cell transforming sequence 2 oncogene |
| CASP8AP2 | 9994 | CASP8 associated protein 2 |
| STK17B | 9262 | Serine/threonine kinase 17b |
| PRKDC | 5591 | Protein kinase, DNA-activated, catalytic polypeptide |
| CRADD | 8738 | CASP2 and RIPK1 domain containing adaptor with death domain |
| BECN1 | 8678 | Beclin 1 (coiled-coil, myosin-like BCL2 interacting protein) |
| CAPN10 | 11132 | Calpain 10 |
| PRUNE2 | 158471 | Prune homolog 2 (Drosophila) |
| SKP2 | 6502 | S-phase kinase-associated protein 2 (p45) |
| ANL1 | 25 | V-abl Abelson murine leukemia viral oncogene homolog 1 |
| CLN3 | 1201 | Ceroid-lipofuscinosis, neuronal 3, juvenile (Batten, Spielmeyer-Vogt |
| | | disease) |
| CTSB | 1508 | Cathepsin B |
| MUC2 | 4583 | Mucin 2, oligomeric mucus/gel-forming |
| NUP62 | 23636 | Nucleoporin 62 kDa |
| APOE | 348 | Apolipoprotein E |

### Set 2:

| Gene Name | EntrezGene ID | Full Name of Gene |
|---|---|---|
| CENPE | 1062 | Centromere protein E, 312 kDa |
| CENPF | 1063 | Centromere protein F, 350/400 ka (mitosin) |
| AURKB | 9212 | Aurora kinase B |
| TTK | 7272 | TTK protein kinase |
| CDCA8 | 55143 | Cell division cycle associated 8 |
| SKP1 | 6500 | S-phase kinase-associated protein 1 |
| CCNA2 | 890 | Cyclin A2 |
| CAMK2G | 818 | Calcium/calmodulin-dependent protein kinase (CaM kinase) |
| | | II gamma |
| INHBA | 3624 | Inhibin, beta A |
| CDC2 | 983 | Cell division cycle 2, G1 to S and G2 to M |
| ERCC6L | 54821 | Excision repair cross-complementing rodent repair |
| | | deficiency, complementation group 6-like |
| BUB1B | 701 | BUB1 budding uninhibited by benzimidazoles 1 homolog |
| | | beta (yeast) |
| NCAPD3 | 23310 | Non-SMC condensin II complex, subunit D3 |
| CDC25A | 993 | Cell division cycle 25 homolog A (S. pombe) |
| DCC1 | 79075 | Defective in sister chromatid cohesion homolog 1 (S. |
| | | cerevisiae) |
| PSMB9 | 5698 | Proteasome (prosome, macropain) subunit, beta type, 9 |
| | | (large multifunctional peptidase 2) |
| DLG7 | 9787 | Discs, large homolog 7 (Drosophila) |
| CHEK1 | 1111 | CHK1 checkpoint homolog (S. pombe) |
| CLASP1 | 23332 | Cytoplasmic linker associated protein 1 |
| SMC2 | 10592 | Structural maintenance of chromosomes 2 |
| ZWINT | 11130 | ZW10 interactor |
| SKP2 | 6502 | S-phase kinase-associated protein 2 (p45) |
| NCAPG | 64151 | Non-SMC condensin I complex, subunit G |
| DBF4 | 10926 | DBF4 homolog (S. cerevisiae) |
| CDC20 | 991 | Cell division cycle 20 homolog (S. cerevisiae) |
| STMN1 | 3925 | Stathmin 1/oncoprotein 18 |
| MDM2 | 4193 | Mdm2, transformed 3T3 cell double minute 2, p53 binding |
| | | protein (mouse) |
| TXNL4B | 54957 | Thioredoxin-like 4B |
| ABL1 | 25 | V-abl Abelson murine leukemia viral oncogene homolog 1 |
| NUMA1 | 4926 | Nuclear mitotic apparatus protein 1 |

### Set 3:

| | EntrezGene | |
|---|---|---|
| Gene Name | ID | Full Name of Gene |
| CCL2 | 6347 | Chemokine (C—C motif) ligand 2 |
| TAP1 | 6890 | Transporter 1, ATP-binding cassette, |
| | | sub-family B (MDR/TAP) |
| CD163 | 9332 | CD163 molecule |
| IFIH1 | 64135 | Interferon induced with helicase C domain 1 |
| SERPINE1 | 5054 | Serpin peptidase inhibitor, clade E (nexin, |
| | | plasminogen activator inhibitor type 1), |
| | | member 1 |
| RSAD2 | 91543 | Radical S-adenosyl methionine domain |
| | | containing 2 |
| DHX58 | 79132 | DEXH (Asp-Glu-X-His) box polypeptide 58 |
| VWF | 7450 | Von Willebrand factor |
| TNFRSF17 | 608 | Tumor necrosis factor receptor superfamily, |
| | | member 17 |
| TNFRSF4 | 7293 | Tumor necrosis factor receptor superfamily, |
| | | member 4 |
| PSG9 | 5678 | Pregnancy specific beta-1-glycoprotein 9 |
| CCR4 | 1233 | Chemokine (C—C motif) receptor 4 |
| FXN | 2395 | Frataxin |
| PARP1 | 142 | Poly (ADP-ribose) polymerase family, |
| | | member 1 |
| C1QB | 713 | Complement component 1, q subcomponent, |
| | | B chain |
| PRKDC | 5591 | Protein kinase, DNA-activated, catalytic |
| | | polypeptide |
| CD38 | 952 | CD38 molecule |
| APOE | 348 | Apolipoprotein E |
| FKBP1A | 2280 | FK506 binding protein 1A, 12 kDa |
| IL4 | 3565 | Interleukin 4 |
| PCSK6 | 5046 | Proprotein convertase subtilisin/kexin type 6 |
| BECN1 | 8678 | Beclin 1 (coiled-coil, myosin-like BCL2 |
| | | interacting protein) |
| PSMB9 | 5698 | Proteasome (prosome, macropain) subunit, |
| | | beta type, 9 (large multifunctional |
| | | peptidase 2) |
| GALNT2 | 2590 | UDP-N-acetyl-alpha-D-galactosamine: |
| | | polypeptide N-acetylgalactosaminyltransferase |
| | | 2 (GalNAc-T2) |
| KLK13 | 26085 | Kallikrein-related peptidase 13 |
| LAX1 | 54900 | Lymphocyte transmembrane adaptor 1 |
| GCH1 | 2643 | GTP cyclohydrolase 1 (dopa-responsive |
| | | dystonia) |
| CLN3 | 1201 | Ceroid-lipofuscinosis, neuronal 3, juvenile |
| | | (Batten, Spielmeyer-Vogt disease) |
| C2 | 717 | Complement component 2 |
| PSG1 | 5669 | Pregnancy specific beta-1-glycoprotein 1 |

### Set 4:

| | EntrezGene | |
|---|---|---|
| Gene Name | ID | Full Name of Gene |
| API5 | 8539 | Apoptosis inhibitor 5 |
| AGT | 183 | Angiotensinogen (serpin peptidase inhibitor, |
| | | clade A, member 8) |
| SAP30BP | 29115 | SAP30 binding protein |
| BNIP3 | 664 | BCL2/adenovirus E1B 19 kDa interacting |
| | | protein 3 |
| GLI3 | 2737 | GLI-Kruppel family member GLI3 (Greig |
| | | cephalopolysyndactyly syndrome) |
| UNC5B | 219699 | Unc-5 homolog B (C. elegans) |
| PDE1B | 5153 | Phosphodiesterase 1B, calmodulin-dependent |
| MSX1 | 4487 | Msh homeobox 1 |
| HIP1 | 3092 | Huntingtin interacting protein 1 |
| PDCD10 | 11235 | Programmed cell death 10 |
| PPARD | 5467 | Peroxisome proliferator-activated receptor |
| | | delta |
| LOC283871 | 283871 | Hypothetical protein LOC283871 |
| RRAGA | 10670 | Ras-related GTP binding A |
| ERBB3 | 2065 | V-erb-b2 erythroblastic leukemia viral |
| | | oncogene homolog 3 (avian) |
| IHPK2 | 51447 | Inositol hexaphosphate kinase 2 |
| EEF1A2 | 1917 | Eukaryotic translation elongation factor 1 |
| | | alpha 2 |
| PERP | 64065 | PERP, TP53 apoptosis effector |
| ATP6AP1 | 537 | ATPase, H+ transporting, lysosomal |
| | | accessory protein 1 |
| ING4 | 51147 | Inhibitor of growth family, member 4 |
| NLRP2 | 55655 | NLR family, pyrin domain containing 2 |
| FXR1 | 8087 | Fragile X mental retardation, autosomal |
| | | homolog 1 |
| C16orf5 | 29965 | Chromosome 16 open reading frame 5 |
| BLCAP | 10904 | Bladder cancer associated protein |
| VEGFA | 7422 | Vascular endothelial growth factor A |
| ESR1 | 2099 | Estrogen receptor 1 |
| TRAF5 | 7188 | TNF receptor-associated factor 5 |
| FIS1 | 51024 | Fission 1 (mitochondrial outer membrane) |
| | | homolog (S. cerevisiae) |
| SFRP1 | 6422 | Secreted frizzled-related protein 1 |
| COMP | 1311 | Cartilage oligomeric matrix protein |
| CDKN2A | 1029 | Cyclin-dependent kinase inhibitor 2A |
| | | (melanoma, p16, inhibits CDK4) |

### Set 5:

| | EntrezGene | |
|---|---|---|
| Gene Name | ID | Full Name of Gene |
| PERP | 64065 | PERP, TP53 apoptosis effector |
| KAL1 | 3730 | Kallmann syndrome 1 sequence |
| EFS | 10278 | Embryonal Fyn-associated substrate |
| CLDN3 | 1365 | Claudin 3 |
| CD36 | 948 | CD36 molecule (thrombospondin receptor) |
| ITGA6 | 3655 | Integrin, alpha 6 |
| CXCL12 | 6387 | Chemokine (C—X—C motif) ligand 12 |
| | | (stromal cell-derived factor 1) |
| PCDHB3 | 56132 | Protocadherin beta 3 |
| RHOB | 388 | Ras homolog gene family, member B |
| ITGB1 | 3688 | Integrin, beta 1 (fibronectin receptor, beta |
| | | polypeptide, antigen CD29 includes MDF2, |
| | | MSK12) |
| GMDS | 2762 | GDP-mannose 4,6-dehydratase |
| DLG1 | 1739 | Discs, large homolog 1 (Drosophila) |
| COL19A1 | 1310 | Collagen, type XIX, alpha 1 |
| SIGLEC8 | 27181 | Sialic acid binding Ig-like lectin 8 |
| PPARD | 5467 | Peroxisome proliferator-activated receptor |
| | | delta |
| IGFALS | 3483 | Insulin-like growth factor binding protein, |
| | | acid labile subunit |
| LAMA4 | 3910 | Laminin, alpha 4 |
| STAB1 | 23166 | Stabilin 1 |
| PTPRM | 5797 | Protein tyrosine phosphatase, receptor type, M |
| SPAM1 | 6677 | Sperm adhesion molecule 1 (PH-20 |
| | | hyaluronidase, zona pellucida binding) |
| AGT | 183 | Angiotensinogen (serpin peptidase inhibitor, |
| | | clade A, member 8) |
| ZYX | 7791 | Zyxin |
| PCDH7 | 5099 | Protocadherin 7 |
| PCDHGB5 | 56101 | Protocadherin gamma subfamily B, 5 |
| MADCAM1 | 8174 | Mucosal vascular addressin cell adhesion |
| | | molecule 1 |
| COMP | 1311 | Cartilage oligomeric matrix protein |
| PVRL2 | 5819 | Poliovirus receptor-related 2 (herpesvirus |
| | | entry mediator B) |
| LAMA5 | 3911 | Laminin, alpha 5 |
| PCDHB17 | 54661 | Protocadherin beta 17 pseudogene |
| ITGA8 | 8516 | Integrin, alpha 8 |

### Set 6:

| | EntrezGene | |
|---|---|---|
| Gene Name | ID | Full Name of Gene |
| PDE1B | 5153 | Phosphodiesterase 1B, calmodulin-dependent |
| ITGA6 | 3655 | Integrin, alpha 6 |
| CCND1 | 595 | Cyclin D1 |
| DEK | 7913 | DEK oncogene (DNA binding) |
| MSX1 | 4487 | Msh homeobox 1 |
| CHAF1B | 8208 | Chromatin assembly factor 1, subunit B (p60) |
| TLK1 | 9874 | Tousled-like kinase 1 |
| SLC25A36 | 55186 | Solute carrier family 25, member 36 |
| RPS6KB1 | 6198 | Ribosomal protein S6 kinase, 70 kDa, |
| | | polypeptide 1 |
| USP1 | 7398 | Ubiquitin specific peptidase 1 |
| AGT | 183 | Angiotensinogen (serpin peptidase inhibitor, |
| | | clade A, member 8) |
| PRKRA | 8575 | Protein kinase, interferon-inducible double |
| | | stranded RNA dependent activator |
| MTMR15 | 22909 | Myotubularin related protein 15 |
| CHRNA3 | 1136 | Cholinergic receptor, nicotinic, alpha 3 |
| C16orf5 | 29965 | Chromosome 16 open reading frame 5 |
| PPARD | 5467 | Peroxisome proliferator-activated receptor |
| | | delta |
| FGB | 2244 | Fibrinogen beta chain |
| ANXA2P2 | 304 | Annexin A2 pseudogene 2 |
| HSPB1 | 3315 | Heat shock 27 kDa protein 1 |
| ANXA2 | 302 | Annexin A2 |
| ESR1 | 2099 | Estrogen receptor 1 |
| SMAD2 | 4087 | SMAD family member 2 |
| STAB1 | 23166 | Stabilin 1 |
| FANCE | 2178 | Fanconi anemia, complementation group E |
| NFATC4 | 4776 | Nuclear factor of activated T-cells, |
| | | cytoplasmic, calcineurin-dependent 4 |
| ERBB3 | 2065 | V-erb-b2 erythroblastic leukemia viral |
| | | oncogene homolog 3 (avian) |
| ERAP1 | 51752 | Endoplasmic reticulum aminopeptidase 1 |
| TOR1B | 27348 | Torsin family 1, member B (torsin B) |
| HPS5 | 11234 | Hermansky-Pudlak syndrome 5 |
| RPA3 | 6119 | Replication protein A3, 14 kDa |

The genes in the marker sets of the present invention are individually known and are individually known to be differentially expressed in tumour cells. How they are differentially expressed and whether their differential expression generally correlates to “good” or “bad” paclitaxel tumour-treating activity can also be determined from publicly available datasets. However, the specific combination of the genes in each marker set of the present invention unexpectedly provides for more robust marker sets having improved accuracy for prediction of whether or not paclitaxel is likely to be effective in treating the tumour. The marker sets of the present invention consisting of the specific combination of genes that gives rise to the improved predictive accuracy may be generated using the Multiple Survival Screening (MSS) method previously developed (Li 2010; Wang 2010).

Paclitaxel is a mitotic inhibitor. It stabilizes microtubules and as a result, interferes with the normal breakdown of microtubules during cell division. Paclitaxel-treated cells have defects in mitotic spindle assembly, chromosome segregation, and cell division. Unlike other tubulin-targeting drugs such as colchicine that inhibit microtubule assembly, paclitaxel stabilizes the microtubule polymer and protects it from disassembly. Chromosomes are thus unable to achieve a metaphase spindle configuration. This blocks progression of mitosis, and prolonged activation of the mitotic checkpoint triggers apoptosis or reversion to the G-phase of the cell cycle without cell division. The ability of paclitaxel to inhibit spindle function is generally attributed to its suppression of microtubule dynamics, however that suppression of dynamics occurs at concentrations lower than those needed to block mitosis. At the higher therapeutic concentrations, paclitaxel appears to suppress microtubule detachment from centrosomes, a process normally activated during mitosis. The binding site for paclitaxel has been identified on the beta-tubulin subunit. Paclitaxel-like drugs have a similar mechanism of action as paclitaxel. Paclitaxel-like drugs include, for example, paclitaxel derivatives (e.g. DHA-paclitaxel, PG-paclitaxel) and other taxanes (e.g. docetaxel).

The sample comprises a sample of the tumour of the patient or an extract thereof, which contains the genes in the marker set or message RNA that hybridizes to the genes in the marker set. Preferably, the sample comprises a sample of the tumour of the patient. The tumour is preferably a breast tumour, ovarian tumor, lung tumour or prostate tumour, more preferably a breast tumour (e.g. estrogen receptor positive (ER+); estrogen receptor negative (ERN triple negative), etc).

Preferably, three marker sets are used together to make predictions. Thus, gene expression profiles of the sample are preferably determined for the genes in each of Sets 1, 2 and 3, or each of Sets 4, 5 and 6. Sets 1, 2 and 3 are particularly useful for determining the effectiveness of paclitaxel for treating ER+ tumours. Sets 4, 5 and 6 are particularly useful for determining the effectiveness of paclitaxel for treating ERN triple negative tumours. In this case, the gene expression profiles are compared to standardized “good” and “bad” profiles of each respective gene marker set to determine whether each of the gene expression profiles predicts that the effectiveness of paclitaxel is “good” or “bad”. If all three marker sets predict that the effectiveness is “good” then the patient is predicted to be a suitable candidate for paclitaxel cancer treatment. If all three marker sets predict that the effectiveness is “bad” then the patient is predicted to be a bad candidate for paclitaxel cancer treatment. If one or two of the marker sets predict that the effectiveness is “good” or one or two of the marker sets predict that the effectiveness is “bad” then the patient is predicted to be an uncertain candidate for paclitaxel cancer treatment. Using all three marker sets improves accuracy of the prediction.

In a particular embodiment, each gene in the gene expression profile has a gene expression value and a modified gene expression profile is obtained by multiplying the gene expression value by its marker-factor. Standardized “good” and “bad” profiles are determined by computing standardized centroids for both “good” and “bad” classes using prediction analysis for microarrays method (Tibshirani 2002). Modified class centroids of the marker set are obtained by multiplying the standardized centroids for each class by the marker-factor. The modified gene expression profile of the sample is compared to each modified class centroid to determine if paclitaxel effectiveness is “good” or “bad”. The class whose centroid is closest to the modified gene expression profile, in Pearson correlation distance, is predicted to be the class for the sample.

Gene expression profiles of a patient's tumour may be readily obtained by any number of methods known in the art, for example microarray analysis, individual gene or RNA screening (e.g. by PCR or real time PCR), diagnostic panels, mini chips, NanoString chips, RNA-seq chips, protein chips, ELISA tests, etc. In a preferred embodiment, a sample may be obtained from a patient by any suitable means, for example, with a syringe or other fluid and/or tissue separation means. The sample may be screened against a microarray on which gene probes of the marker sets are printed. An output of the gene expression profile of the sample is preferably obtained before comparing the gene expression profile to the standardized “good” and “bad” profiles of the marker set. To obtain the output, message RNA in the sample may be hybridized to the genes on the microarray, the hybridized microarray may be scanned to get all the readouts of marker genes for the sample, the readouts may be normalized and the gene expression profile of the marker set for the sample is thereby obtained. Detailed information for making microarray gene chip, scanning and normalization of array data is generally known in the art and can be found in the publicly available literature (http://en.wikipedia.org/wiki/DNA_microarray). It is also possible to obtain the gene expression profile by RNA-sequencing and related sequencing technologies as these technologies become more accessible (http://en.wikipedia.org/wiki/RNA-Seq).

In another embodiment, kits or commercial packages are provided, which comprise gene probes for each of the genes in a gene marker set of the present invention along with instructions for obtaining a gene expression profile of a sample for the gene marker set. The kit or commercial package may further comprise instructions for comparing the gene expression profile of the sample to standardized “good” and “bad” profiles of the marker set to determine whether the gene expression profile of the sample predicts that paclitaxel effectiveness is “good” or “bad”. Preferably, the kit or commercial package comprises gene probes for at least three gene marker sets of the present invention. The kit or commercial package may further comprise means for obtaining a sample of a tumour having message RNA therein from a patient, for example suitable syringes, fluid and/or tissue separation means, etc. In addition to the gene probes, the kit or commercial package may further comprise reagents and/or equipment useful for screening the sample against the gene probes for obtaining the gene expression profile of the sample. Various standard elements of such kits or commercial packages are generally known in the art.

Further features of the invention will be described or will become apparent in the course of the following detailed description.

### DESCRIPTION OF PREFERRED EMBODIMENTS

### Example 1

### Generation of Paclitaxel Response Marker Sets for ER+ Breast Cancer

To develop ER+ cancer marker sets of the present invention, the Multiple Survival Screening (MSS) method (Li 2010; Wang 2010) was used. In applying this method, a training set of 260 ER+ breast cancer samples was selected from a public metadata set (GEO GSE4779, GSE20194, GSE20271, GSE22093 and GSE23988). Each patient has been treated with paclitaxel and followed-up pathologically to determine who is responsive to the treatment. The primary tumors prior to any drug treatment have been microarray profiled. The datasets contain information about gene expression profiles for patient primary tumours and the information of response/non-response for paclitaxel treatment for each patient. Datasets identify whether each of these genes is up-regulated or down-regulated in tumours and correlates these genes with responsiveness to paclitaxel treatment (i.e. “good” vs. “bad”).

100 samples from the datasets were randomly selected in which 70 were samples that did not respond to paclitaxel treatment (“bad”) and 30 were samples that did respond to paclitaxel treatment (“good”). Array-wide single-gene based clustering (using fuzzy clustering method, http://stat.ethz.ch/R-manual/R-patched/library/cluster/html/fanny.html) of responsive/non-responsive was conducted to obtain effectiveness genes, which are genes whose differential expression values are correlated with effective paclitaxel treatment. It is not relevant whether the expression of each gene is upregulated or downregulated so long as the differential expression is correlated to effective paclitaxel treatment. Selection of samples and array-wide single-gene based clustering analyses (using fuzzy clustering method, http://stat.ethz.ch/R-manual/R-patched/library/cluster/html/fanny.html) were repeated 100 times, and the effectiveness genes (which have P value <0.05 in more than 75 out of the 100 times) from each of the 100 repetitions were merged.

Using the effectiveness gene set, Gene Ontology (GO) analysis (using GO annotation software, David, http://david.abcc.ncifcrf.gov/) was performed to identify only those genes that belong to GO terms that are known to be associated with cancer, such as apoptosis, response to wounding, DNA replication and transcription repair, mitosis and immune response. Table 1 lists the ER+ cancer-related GO term gene sets. Two million distinct random-gene-sets were generated by randomly picking 30 genes from each ER+ cancer-related GO term gene set.

TABLE 1

| GO Term | Number of genes |
|---|---|
| Apoptosis | 68 |
| Response to wounding | 60 |
| DNA replication and transcription repair | 53 |
| Mitosis | 63 |
| Immune response | 63 |

Of 83 samples (58 with no response to paclitaxel treatment and 25 that responded to paclitaxel treatment) selected from the dataset to form the training set, 36 random datasets were generated. For a given GO term gene set, paclitaxel effectiveness screening was then conducted using the 2 million random-gene-sets against all the 36 random datasets. For each random dataset, the statistical significance of the correlation between the expression values of each random-gene-set (30 genes) and paclitaxel effectiveness status (“good” or “bad”) was examined by fuzzy clustering analysis (using fuzzy clustering method, http://stat.ethz.ch/R-manual/R-patched/library/cluster/html/fanny.html). If the P value was less than a cut-off for an effectiveness screening using one random-gene-set against one random dataset, that random-gene-set was said to have passed. When a few thousands of random-gene-sets had passed 32 or more random datasets (the detailed parameters are shown in Table 2), the random-gene-sets that had passed were retained for further analysis. The genes in the retained random-gene-sets were then ranked based on their frequency of appearance in the passed random-gene-sets. The top 30 genes were chosen as a potential-marker-set. A similar effectiveness screening of random-gene-sets against random datasets was performed for each of the other selected GO term gene sets. Only apoptosis, mitosis and immune response GO term gene sets were used to generate the ER+ marker sets.

TABLE 2
Parameters for Screening of the Marker Sets

| | Number of Passed | Number of Passed | Cut-off |
|---|---|---|---|
| | Sample Sets | Gene Sets | P value |
| Apoptosis | 32 | 1586 | 0.01 |
| Mitosis | 32 | 4370 | 0.005 |
| Immune response | 34 | 2959 | 0.05 |

For each GO term gene set used, another 1 million distinct random-gene-sets were generated and the clustering process using the random datasets mentioned above was repeated. If the gene members for the top 30 were substantially the same as those in the potential-marker-set generated by the first screening, then the potential-marker-set is stable and can be used as a real ER+ cancer marker set. If the genes for the two potential marker sets were not substantially the same, then these GO term genes are unsuitable for finding a real marker set and the potential marker set was dropped from further analysis.

In this way, three ER+ cancer marker sets were generated having stable signatures, one related to apoptosis (Set 1), one related to mitosis (Set 2) and one related to immune response (Set 3). The genes, EntrezGene ID and full names of the genes in each of the three marker sets are given above. More details of each gene, including the nucleotide sequence of each gene, are known in the art and may be conveniently found in the National Center for Biotechnology Information (NCBI) Databases at http://www.ncbi.nlm.nih.gov/.

### Example 2

### Generation of Paclitaxel Response Marker Sets for ERN Breast Cancer

To develop ERN (estrogen receptor negative) cancer marker sets of the present invention, the Multiple Survival Screening (MSS) method (Li 2010; Wang 2010) was used. In applying this method, a training set of 202 ERN breast cancer samples was selected from GSE25066 dataset (Hatzis 2011). The dataset contains information which is the same as those described above (the ER+ datasets). 153 samples from the dataset were randomly selected in which 100 were samples that did not respond to paclitaxel treatment (“bad”) and 53 were samples that did respond to paclitaxel treatment (“good”). Array-wide single-gene based fuzzy clustering (using fuzzy clustering method, http://stat.ethz.ch/R-manual/R-patched/library/cluster/html/fanny.html) screening of responsive/non-responsive samples was performed to obtain effectiveness genes, which are genes whose differential expression values are correlated with effective paclitaxel treatment. It is not relevant whether the expression of each gene is upregulated or downregulated so long as the differential expression is correlated to effective paclitaxel treatment. Selection of samples and array-wide screening were repeated 3 times, and effectiveness genes (P value <0.05) from each of the 3 repetitions were merged. Using the effectiveness gene set, Gene Ontology (GO) analysis (using GO annotation software, David, http://david.abcc.ncifcrf.gov/) was performed to identify only those genes that belong to GO terms that are known to be associated with cancer, such as apoptosis, cell cycle, cell adhesion, response, DNA repair & replication and mitosis. Table 3 lists the ERN cancer-related GO term gene sets. Two million distinct random-gene-sets were generated by randomly picking 30 genes from each ERN cancer-related GO term gene set.

TABLE 3

| GO Term | Number of genes |
|---|---|
| Apoptosis | 82 |
| Cell cycle | 88 |
| Cell adhesion | 47 |
| Response to stimulus | 61 |
| DNA repair & replication | 53 |
| Mitosis | 45 |

Of 152 samples (99 with no response to paclitaxel treatment and 53 that responded to paclitaxel treatment) selected from the dataset to form the training set, 36 random datasets were generated. For a given GO term gene set, paclitaxel effectiveness screening was then conducted using the 1 million random-gene-sets against all the 36 random datasets. For each random dataset, the statistical significance of the correlation between the expression values of each random-gene-set (30 genes) and paclitaxel effectiveness status (“good” or “bad”) was examined by fuzzy clustering analysis (using fuzzy clustering method, http://stat.ethz.ch/R-manual/R-patched/library/cluster/html/fanny.html). If the P value was less than a cut-off for an effectiveness screening using one random-gene-set against one random dataset, that random-gene-set was said to have passed. When a few thousands of random-gene-sets had passed 32 or more random datasets (the detailed parameters are shown in Table 4), the random-gene-sets that had passed were retained for further analysis. The genes in the retained random-gene-sets were then ranked based on their frequency of appearance in the passed random-gene-sets. The top 30 genes were chosen as a potential-marker-set. A similar effectiveness screening of random-gene-sets against random datasets was performed for each of the other selected GO term gene sets. Only apoptosis, cell adhesion and response GO term gene sets were used to generate the ERN marker sets.

TABLE 4
Parameters for Screening of the Marker Sets

| | Number of Passed | Number of Passed | Cut-off |
|---|---|---|---|
| | Sample Sets | Gene Sets | P value |
| Apoptosis | 36 | 4454 | 0.005 |
| Cell adhesion | 36 | 5779 | 0.05 |
| Response to | 36 | 10682 | 0.005 |
| stimulus | | | |

For each GO term gene set used, another 1 million distinct random-gene-sets were generated and the survival screening process using the random datasets mentioned above was repeated. If the gene members for the top 30 were substantially the same as those in the potential-marker-set generated by the first screening, then the potential-marker-set is stable and can be used as a real ERN cancer marker set. If the genes for the two potential marker sets were not substantially the same, then these GO term genes are unsuitable for finding a real marker set and the potential marker set was dropped from further analysis.

In this way, three ERN cancer marker sets were generated having stable signatures, one related to apoptosis (Set 4), one related to cell adhesion (Set 5) and one related to response to stimulus (Set 6). The genes, EntrezGene ID and full names of the genes in each of the three marker sets are given above. More details of each gene, including the nucleotide sequence of each gene, are known in the art and may be conveniently found in the National Center for Biotechnology Information (NCBI) Databases at http://www.ncbi.nlm.nih.gov/.

### Example 3

### Validating Effectiveness of the Marker Sets in Predicting Paclitaxel Effectiveness for Treating Breast Cancer

The effectiveness of the marker sets generated in Examples 1 and 2 was validated against datasets containing breast cancer gene expression data from sample populations. Sets 1, 2 and 3 from Example 1 were validated against metadata from public data (GSE4779, GSE20194, GSE20271, GSE22093 and GSE23988) and against the GSE25066 dataset (Hatzis 2011). Sets 4, 5 and 6 from Example 2 were validated against the GSE25066 dataset (ERN, 87% triple negative) (Hatzis 2011), the GSE20174 dataset (triple negative) (Zeidler-Erdely 2010), and the GSE20194 dataset (triple negative) (Popovici 2010; Shi 2010).

To perform the validation for a given test dataset containing ‘n’ samples, the gene expression profile of the marker set was extracted. For each gene expression value its marker-factor was multiplied to obtain a modified gene expression profile of the testing sample. Standardized centroids were computed for both “good” and “bad” classes from n−1 samples for the marker set using the Prediction Analysis for Microarrays (PAM) method (Tibshirani 2002). The marker-factor of each gene was multiplied to the class centroids to get modified class centroids of the marker set. For predicting the paclitaxel response of the targeted testing sample using the marker set, the modified gene expression profile of the sample was compared to each of these modified class centroids. The class whose centroid that it is closest to, in Pearson correlation distance, is the predicted class for that sample. If the sample is predicted to be unresponsive to paclitaxel treatment (i.e. “bad”), it is denoted as 0, otherwise it is denoted as 1. If all three marker sets (Sets 1, 2 and 3, or Sets 4, 5 and 6) predict that a particular sample is unresponsive to paclitaxel (i.e. denoted as 0 for all 3 marker sets), the sample is assigned to a paclitaxel unresponsive group (i.e. “bad”). If all three marker sets predict that a particular sample is responsive to paclitaxel (i.e. denoted as 1 for all 3 marker sets), the sample is assigned to a paclitaxel responsive group (i.e. “good”). If a sample is not assigned to either of these groups, it is assigned to an indeterminate group.

This validation process was carried out in each of the test datasets. Table 5 shows the accuracy for Sets 1, 2 and 3 in predicting the paclitaxel unresponsive group in the metadata from public data dataset and the GSE25066 dataset. Table 6 shows the accuracy for Sets 4, 5 and 6 in predicting the paclitaxel unresponsive group in the GSE25066 dataset, the GSE20174 dataset and the GSE20194 dataset. The accuracy of the marker sets against the test datasets is remarkably high, and much higher than the 50-60% that can be achieved using current prior art marker sets (Hatzis 2011).

TABLE 5
Accuracy of Sets 1, 2 and 3

| | | Accuracy (paclitaxel |
|---|---|---|
| Dataset | No. of Samples | unresponsive group) |
| Metadata from public data | 260 | 95.4% |
| (training part: GSE4779, | | |
| GSE20194, GSE20271, | | |
| GSE22093 and GSE23988) | | |
| Metadata from public data | 111 | 97.2% |
| (test part: GSE4779, | | |
| GSE20194, GSE20271, | | |
| GSE22093 and GSE23988) | | |
| GSE25066 | 290 | 96.3% |

TABLE 6
Accuracy of Sets 4, 5 and 6

| | | Accuracy (paclitaxel |
|---|---|---|
| Dataset | No. of Samples | unresponsive group) |
| GSE25066 (training) | 202 | 91% |
| GSE20174 | 59 | 91% |
| GSE20194 | 70 | 88% |

### REFERENCES

The contents of the entirety of each of which are incorporated by this reference.
Cui Q, Ma Y, Jaramillo M, Bari H, Awan A, Yang S, Zhang S, Liu L, Lu M, O'Connor-McCourt M, Purisima E O, Wang E. (2007) A map of human cancer signaling. Molecular Systems Biology. 3:152, 13 pages.
Fuzzy Analysis Clustering version 1.14.0. (2011) http://stat.ethz.ch/R-manual/R-patched/library/cluster/html/fanny.html.
GO annotation software, David. http://david.abcc.ncifcrf.gov/.
Hatzis C, et al. (2011) A Genomic Predictor of Response and Survival Following Taxane-Anthracycline Chemotherapy for Invasive Breast Cancer. JAMA. 305(18): 1873-1881.
La Thangue NB, Kerr D J. (2011) Predictive biomarkers: a paradigm shift towards personalized cancer medicine. Nat. Rev. Clin. Oncol. 8, 587-596.
Li J, Lenferink AEG, Deng Y, Collins C, Cui Q, Purisima EO, O'Connor-McCourt M D, Wang E. (2010) Identification of high-quality cancer prognostic markers and metastasis network modules. Nature Communications. 1:34, DOI: 10.1038/ncomms1033.
National Center for Biotechnology Information (NCBI) Databases. http://www.ncbi.nlm.nih.gov/.
Popovici V, Chen W, Gallas B G, Hatzis C, et al. (2010) Effect of training-sample size and classification difficulty on the accuracy of genomic predictors. Breast Cancer Res. 12(1), R5.
Sarker D, Workman P. (2007) Pharmacodynamic biomarkers for molecular cancer therapeutics. Adv. Cancer Res. 96, 213-268.
Shi L, Campbell G, Jones W D, Campagne F, et al. (2010) The MicroArray Quality Control (MAQC)-II study of common practices for the development and validation of microarray-based predictive models. Nat Biotechnol. 28(8), 827-38.
Tibshirani R, Hastie T, Narasimhan B, Chu G. (2002) Diagnosis of multiple cancer types by shrunken centroids of gene expression. PNAS. 99, 6567-6572.
Wang E, Li J, Deng Y, Lenferink AEG, O'Connor-McCourt M D, Purisima EO. (2010) Process for Tumour Characteristic and Marker Set Identification, Tumour Classification and Marker Sets for Cancer. International Patent Application WO 2010/118520 published Oct. 21, 2010.
Wikipedia, the free encyclopedia. (2010a) DNA Microarray. http://en.wikipedia.org/wiki/DNA_microarray.
Wikipedia, the free encyclopedia. (2010b) RNA-Seq. http://en.wikipedia.org/wiki/RNA-Seq.
Zeidler-Erdely P C, Kashon M L, Li S, Antonini J M. (2010) Response of the mouse lung transcriptome to welding fume: effects of stainless and mild steel fumes on lung gene expression in NJ and C57BL/6J mice. Respir Res. 11(1), 70 (18 pages).

Other advantages that are inherent to the structure are obvious to one skilled in the art. The embodiments are described herein illustratively and are not meant to limit the scope of the invention as claimed. Variations of the foregoing embodiments will be evident to a person of ordinary skill and are intended by the inventor to be encompassed by the following claims.

1. A method of determining likelihood that a tumour in a patient would be treatable with paclitaxel or a paclitaxel-like drug, the method comprising:
(a) obtaining a gene expression list of a sample of the tumour or an extract of the tumour having message RNA therein of the patient;
(b) determining a gene expression profile of the sample from the gene expression list for genes of a gene marker set; and,
(c) comparing the gene expression profile of the sample to standardized “good” and “bad” profiles of the marker set to determine whether the gene expression profile of the sample predicts that the tumour is treatable or not treatable with paclitaxel or a paclitaxel-like drug, wherein “good” indicates that the tumour is likely treatable with paclitaxel or a paclitaxel-like drug and “bad” indicates that the tumour is not likely treatable with paclitaxel or a paclitaxel-like drug,
and the gene marker set is Set 1, Set 2, Set 3, Set 4, Set 5, Set 6 or a combination thereof, wherein
Set 1 consists of:

| | EntrezGene | |
|---|---|---|
| Gene | ID | Full Name of Gene |
| HELLS | 3070 | Helicase, lymphoid-specific |
| CDC2 | 983 | Cell division cycle 2, G1 to S and G2 to M |
| PLEKHF1 | 79156 | Pleckstrin homology domain containing, |
| | | family F (with FYVE domain) member 1 |
| IGFBP3 | 3486 | Insulin-like growth factor binding protein 3 |
| CASP3 | 836 | Caspase 3, apoptosis-related cysteine peptidase |
| HRK | 8739 | Harakiri, BCL2 interacting protein (contains |
| | | only BH3 domain) |
| PCSK6 | 5046 | Proprotein convertase subtilisin/kexin type 6 |
| PLAGL1 | 5325 | Pleiomorphic adenoma gene-like 1 |
| NME5 | 8382 | Non-metastatic cells 5, protein expressed in |
| | | (nucleoside-diphosphate kinase) |
| PROP1 | 5626 | PROP paired-like homeobox 1 |
| NOD2 | 64127 | Nucleotide-binding oligomerization domain |
| | | containing 2 |
| CD38 | 952 | CD38 molecule |
| ATP7A | 538 | ATPase, Cu++ transporting, alpha polypeptide |
| | | (Menkes syndrome) |
| INDO | 3620 | Indoleamine-pyrrole 2,3 dioxygenase |
| PIM2 | 11040 | Pim-2 oncogene |
| ECT2 | 1894 | Epithelial cell transforming sequence 2 |
| | | oncogene |
| CASP8AP2 | 9994 | CASP8 associated protein 2 |
| STK17B | 9262 | Serine/threonine kinase 17b |
| PRKDC | 5591 | Protein kinase, DNA-activated, catalytic |
| | | polypeptide |
| CRADD | 8738 | CASP2 and RIPK1 domain containing adaptor |
| | | with death domain |
| BECN1 | 8678 | Beclin 1 (coiled-coil, myosin-like BCL2 |
| | | interacting protein) |
| CAPN10 | 11132 | Calpain 10 |
| PRUNE2 | 158471 | Prune homolog 2 (Drosophila) |
| SKP2 | 6502 | S-phase kinase-associated protein 2 (p45) |
| ANL1 | 25 | V-abl Abelson murine leukemia viral oncogene |
| | | homolog 1 |
| CLN3 | 1201 | Ceroid-lipofuscinosis, neuronal 3, juvenile |
| | | (Batten, Spielmeyer-Vogt disease) |
| CTSB | 1508 | Cathepsin B |
| MUC2 | 4583 | Mucin 2, oligomeric mucus/gel-forming |
| NUP62 | 23636 | Nucleoporin 62 kDa |
| APOE | 348 | Apolipoprotein E |

Set 2 consists of:

| | EntrezGene | |
|---|---|---|
| Gene Name | ID | Full Name of Gene |
| CENPE | 1062 | Centromere protein E, 312 kDa |
| CENPF | 1063 | Centromere protein F, 350/400 ka (mitosin) |
| AURKB | 9212 | Aurora kinase B |
| TTK | 7272 | TTK protein kinase |
| CDCA8 | 55143 | Cell division cycle associated 8 |
| SKP1 | 6500 | S-phase kinase-associated protein 1 |
| CCNA2 | 890 | Cyclin A2 |
| CAMK2G | 818 | Calcium/calmodulin-dependent protein kinase |
| | | (CaM kinase) II gamma |
| INHBA | 3624 | Inhibin, beta A |
| CDC2 | 983 | Cell division cycle 2, G1 to S and G2 to M |
| ERCC6L | 54821 | Excision repair cross-complementing rodent |
| | | repair deficiency, complementation group 6-like |
| BUB1B | 701 | BUB1 budding uninhibited by benzimidazoles |
| | | 1 homolog beta (yeast) |
| NCAPD3 | 23310 | Non-SMC condensin II complex, subunit D3 |
| CDC25A | 993 | Cell division cycle 25 homolog A (S. pombe) |
| DCC1 | 79075 | Defective in sister chromatid cohesion homolog |
| | | 1 (S. cerevisiae) |
| PSMB9 | 5698 | Proteasome (prosome, macropain) subunit, |
| | | beta type, 9 (large multifunctional peptidase 2) |
| DLG7 | 9787 | Discs, large homolog 7 (Drosophila) |
| CHEK1 | 1111 | CHK1 checkpoint homolog (S. pombe) |
| CLASP1 | 23332 | Cytoplasmic linker associated protein 1 |
| SMC2 | 10592 | Structural maintenance of chromosomes 2 |
| ZWINT | 11130 | ZW10 interactor |
| SKP2 | 6502 | S-phase kinase-associated protein 2 (p45) |
| NCAPG | 64151 | Non-SMC condensin I complex, subunit G |
| DBF4 | 10926 | DBF4 homolog (S. cerevisiae) |
| CDC20 | 991 | Cell division cycle 20 homolog (S. cerevisiae) |
| STMN1 | 3925 | Stathmin 1/oncoprotein 18 |
| MDM2 | 4193 | Mdm2, transformed 3T3 cell double minute 2, |
| | | p53 binding protein (mouse) |
| TXNL4B | 54957 | Thioredoxin-like 4B |
| ABL1 | 25 | V-abl Abelson murine leukemia viral oncogene |
| | | homolog 1 |
| NUMA1 | 4926 | Nuclear mitotic apparatus protein 1 |

Set 3 consists of:

| | EntrezGene | |
|---|---|---|
| Gene Name | ID | Full Name of Gene |
| CCL2 | 6347 | Chemokine (C—C motif) ligand 2 |
| TAP1 | 6890 | Transporter 1, ATP-binding cassette, sub- |
| | | family B (MDR/TAP) |
| CD163 | 9332 | CD163 molecule |
| IFIH1 | 64135 | Interferon induced with helicase C domain 1 |
| SERPINE1 | 5054 | Serpin peptidase inhibitor, clade E (nexin, |
| | | plasminogen activator inhibitor type 1), |
| | | member 1 |
| RSAD2 | 91543 | Radical S-adenosyl methionine domain |
| | | containing 2 |
| DHX58 | 79132 | DEXH (Asp-Glu-X-His) box polypeptide 58 |
| VWF | 7450 | Von Willebrand factor |
| TNFRSF17 | 608 | Tumor necrosis factor receptor superfamily, |
| | | member 17 |
| TNFRSF4 | 7293 | Tumor necrosis factor receptor superfamily, |
| | | member 4 |
| PSG9 | 5678 | Pregnancy specific beta-1-glycoprotein 9 |
| CCR4 | 1233 | Chemokine (C—C motif) receptor 4 |
| FXN | 2395 | Frataxin |
| PARP1 | 142 | Poly (ADP-ribose) polymerase family, |
| | | member 1 |
| C1QB | 713 | Complement component 1, q subcomponent, |
| | | B chain |
| PRKDC | 5591 | Protein kinase, DNA-activated, catalytic |
| | | polypeptide |
| CD38 | 952 | CD38 molecule |
| APOE | 348 | Apolipoprotein E |
| FKBP1A | 2280 | FK506 binding protein 1A, 12 kDa |
| IL4 | 3565 | Interleukin 4 |
| PCSK6 | 5046 | Proprotein convertase subtilisin/kexin type 6 |
| BECN1 | 8678 | Beclin 1 (coiled-coil, myosin-like BCL2 |
| | | interacting protein) |
| PSMB9 | 5698 | Proteasome (prosome, macropain) subunit, |
| | | beta type, 9 (large multifunctional peptidase 2) |
| GALNT2 | 2590 | UDP-N-acetyl-alpha-D-galactosamine: |
| | | polypeptide N-acetylgalactosaminyltransferase |
| | | 2 (GalNAc-T2) |
| KLK13 | 26085 | Kallikrein-related peptidase 13 |
| LAX1 | 54900 | Lymphocyte transmembrane adaptor 1 |
| GCH1 | 2643 | GTP cyclohydrolase 1 (dopa-responsive |
| | | dystonia) |
| CLN3 | 1201 | Ceroid-lipofuscinosis, neuronal 3, juvenile |
| | | (Batten, Spielmeyer-Vogt disease) |
| C2 | 717 | Complement component 2 |
| PSG1 | 5669 | Pregnancy specific beta-1-glycoprotein 1 |

Set 4 consists of:

| | EntrezGene | |
|---|---|---|
| Gene Name | ID | Full Name of Gene |
| API5 | 8539 | Apoptosis inhibitor 5 |
| AGT | 183 | Angiotensinogen (serpin peptidase inhibitor, |
| | | clade A, member 8) |
| SAP30BP | 29115 | SAP30 binding protein |
| BNIP3 | 664 | BCL2/adenovirus E1B 19 kDa interacting |
| | | protein 3 |
| GLI3 | 2737 | GLI-Kruppel family member GLI3 (Greig |
| | | cephalopolysyndactyly syndrome) |
| UNC5B | 219699 | Unc-5 homolog B (C. elegans) |
| PDE1B | 5153 | Phosphodiesterase 1B, calmodulin-dependent |
| MSX1 | 4487 | Msh homeobox 1 |
| HIP1 | 3092 | Huntingtin interacting protein 1 |
| PDCD10 | 11235 | Programmed cell death 10 |
| PPARD | 5467 | Peroxisome proliferator-activated receptor |
| LOC283871 | 283871 | Hypothetical protein LOC283871delta |
| RRAGA | 10670 | Ras-related GTP binding A |
| ERBB3 | 2065 | V-erb-b2 erythroblastic leukemia viral |
| | | oncogene homolog 3 (avian) |
| IHPK2 | 51447 | Inositol hexaphosphate kinase 2 |
| EEF1A2 | 1917 | Eukaryotic translation elongation factor 1 |
| | | alpha 2 |
| PERP | 64065 | PERP, TP53 apoptosis effector |
| ATP6AP1 | 537 | ATPase, H+ transporting, lysosomal accessory |
| | | protein 1 |
| ING4 | 51147 | Inhibitor of growth family, member 4 |
| NLRP2 | 55655 | NLR family, pyrin domain containing 2 |
| FXR1 | 8087 | Fragile X mental retardation, autosomal |
| | | homolog 1 |
| C16orf5 | 29965 | Chromosome 16 open reading frame 5 |
| BLCAP | 10904 | Bladder cancer associated protein |
| VEGFA | 7422 | Vascular endothelial growth factor A |
| ESR1 | 2099 | Estrogen receptor 1 |
| TRAF5 | 7188 | TNF receptor-associated factor 5 |
| FIS1 | 51024 | Fission 1 (mitochondrial outer membrane) |
| | | homolog (S. cerevisiae) |
| SFRP1 | 6422 | Secreted frizzled-related protein 1 |
| COMP | 1311 | Cartilage oligomeric matrix protein |
| CDKN2A | 1029 | Cyclin-dependent kinase inhibitor 2A |
| | | (melanoma, p16, inhibits CDK4) |

Set 5 consists of:

| | EntrezGene | |
|---|---|---|
| Gene Name | ID | Full Name of Gene |
| PERP | 64065 | PERP, TP53 apoptosis effector |
| KAL1 | 3730 | Kallmann syndrome 1 sequence |
| EFS | 10278 | Embryonal Fyn-associated substrate |
| CLDN3 | 1365 | Claudin 3 |
| CD36 | 948 | CD36 molecule (thrombospondin receptor) |
| ITGA6 | 3655 | Integrin, alpha 6 |
| CXCL12 | 6387 | Chemokine (C—X—C motif) ligand 12 |
| | | (stromal cell-derived factor 1) |
| PCDHB3 | 56132 | Protocadherin beta 3 |
| RHOB | 388 | Ras homolog gene family, member B |
| ITGB1 | 3688 | Integrin, beta 1 (fibronectin receptor, beta |
| | | polypeptide, antigen CD29 includes MDF2, |
| | | MSK12) |
| GMDS | 2762 | GDP-mannose 4,6-dehydratase |
| DLG1 | 1739 | Discs, large homolog 1 (Drosophila) |
| COL19A1 | 1310 | Collagen, type XIX, alpha 1 |
| SIGLEC8 | 27181 | Sialic acid binding Ig-like lectin 8 |
| PPARD | 5467 | Peroxisome proliferator-activated receptor |
| | | delta |
| IGFALS | 3483 | Insulin-like growth factor binding protein, |
| | | acid labile subunit |
| LAMA4 | 3910 | Laminin, alpha 4 |
| STAB1 | 23166 | Stabilin 1 |
| PTPRM | 5797 | Protein tyrosine phosphatase, receptor type, M |
| SPAM1 | 6677 | Sperm adhesion molecule 1 (PH-20 |
| | | hyaluronidase, zona pellucida binding) |
| AGT | 183 | Angiotensinogen (serpin peptidase inhibitor, |
| | | clade A, member 8) |
| ZYX | 7791 | Zyxin |
| PCDH7 | 5099 | Protocadherin 7 |
| PCDHGB5 | 56101 | Protocadherin gamma subfamily B, 5 |
| MADCAM1 | 8174 | Mucosal vascular addressin cell adhesion |
| | | molecule 1 |
| COMP | 1311 | Cartilage oligomeric matrix protein |
| PVRL2 | 5819 | Poliovirus receptor-related 2 (herpesvirus |
| | | entry mediator B) |
| LAMA5 | 3911 | Laminin, alpha 5 |
| PCDHB17 | 54661 | Protocadherin beta 17 pseudogene |
| ITGA8 | 8516 | Integrin, alpha 8 |

Set 6 consists of:

| | EntrezGene | |
|---|---|---|
| Gene Name | ID | Full Name of Gene |
| PDE1B | 5153 | Phosphodiesterase 1B, calmodulin-dependent |
| ITGA6 | 3655 | Integrin, alpha 6 |
| CCND1 | 595 | Cyclin D1 |
| DEK | 7913 | DEK oncogene (DNA binding) |
| MSX1 | 4487 | Msh homeobox 1 |
| CHAF1B | 8208 | Chromatin assembly factor 1, subunit B (p60) |
| TLK1 | 9874 | Tousled-like kinase 1 |
| SLC25A36 | 55186 | Solute carrier family 25, member 36 |
| RPS6KB1 | 6198 | Ribosomal protein S6 kinase, 70 kDa, |
| | | polypeptide 1 |
| USP1 | 7398 | Ubiquitin specific peptidase 1 |
| AGT | 183 | Angiotensinogen (serpin peptidase inhibitor, |
| | | clade A, member 8) |
| PRKRA | 8575 | Protein kinase, interferon-inducible double |
| | | stranded RNA dependent activator |
| MTMR15 | 22909 | Myotubularin related protein 15 |
| CHRNA3 | 1136 | Cholinergic receptor, nicotinic, alpha 3 |
| C16orf5 | 29965 | Chromosome 16 open reading frame 5 |
| PPARD | 5467 | Peroxisome proliferator-activated receptor |
| | | delta |
| FGB | 2244 | Fibrinogen beta chain |
| ANXA2P2 | 304 | Annexin A2 pseudogene 2 |
| HSPB1 | 3315 | Heat shock 27 kDa protein 1 |
| ANXA2 | 302 | Annexin A2 |
| ESR1 | 2099 | Estrogen receptor 1 |
| SMAD2 | 4087 | SMAD family member 2 |
| STAB1 | 23166 | Stabilin 1 |
| FANCE | 2178 | Fanconi anemia, complementation group E |
| NFATC4 | 4776 | Nuclear factor of activated T-cells, |
| | | cytoplasmic, calcineurin-dependent 4 |
| ERBB3 | 2065 | V-erb-b2 erythroblastic leukemia viral |
| | | oncogene homolog 3 (avian) |
| ERAP1 | 51752 | Endoplasmic reticulum aminopeptidase 1 |
| TOR1B | 27348 | Torsin family 1, member B (torsin B) |
| HPS5 | 11234 | Hermansky-Pudlak syndrome 5 |
| RPA3 | 6119 | Replication protein A3, 14 kDa |

2. The method according to claim 1, wherein the tumour is a breast tumour, ovarian tumour, lung tumour or prostate tumor.

3. The method according to claim 1, wherein the tumour is a breast tumour.

4. The method according to any one of claims 1 to 3, wherein gene expression profiles of the sample are determined for the genes in each of Sets 1, 2 and 3 and the gene expression profiles are compared to standardized “good” and “bad” profiles of each respective gene marker set to determine whether each of the gene expression profiles predicts that the tumour is treatable or not treatable with paclitaxel or a paclitaxel-like drug, whereby if all three marker sets predict that the tumour is treatable then the patient is predicted to likely benefit from paclitaxel or paclitaxel-like drug treatment, if all three marker sets predict that the tumour is untreatable then the patient is predicted to unlikely benefit from paclitaxel or a paclitaxel-like drug treatment and if one or two of the marker sets predict that the tumour is treatable or one or two of the marker sets predict that the tumour is untreatable then it is indeterminate whether the patient would benefit from paclitaxel or a paclitaxel-like drug treatment.

5. The method according to claim 4, wherein the tumour is an estrogen receptor positive (ER+) tumour.

6. The method according to any one of claims 1 to 3, wherein gene expression profiles of the sample are determined for the genes in each of Sets 4, 5 and 6 and the gene expression profiles are compared to standardized “good” and “bad” profiles of each respective gene marker set to determine whether each of the gene expression profiles predicts that the tumour is treatable or not treatable with paclitaxel or a paclitaxel-like drug, whereby if all three marker sets predict that the tumour is treatable then the patient is predicted to likely benefit from paclitaxel or paclitaxel-like drug treatment, if all three marker sets predict that the tumour is untreatable then the patient is predicted to unlikely benefit from paclitaxel or a paclitaxel-like drug treatment and if one or two of the marker sets predict that the tumour is treatable or one or two of the marker sets predict that the tumour is untreatable then it is indeterminate whether the patient would benefit from paclitaxel or a paclitaxel-like drug treatment.

7. The method according to claim 6, wherein the tumour is an estrogen receptor negative (ERN triple negative) tumor.

8. A method of screening a chemical compound as a drug candidate with paclitaxel-like tumour-treating activity, the method comprising:
(a) determining a gene expression profile for genes of a gene marker set of a tumor sample treated with the chemical compound; and,
(b) comparing the gene expression profile of the sample to standardized “good” and “bad” profiles of the marker set to determine whether the gene expression profile of the sample predicts that the chemical compound would have paclitaxel-like tumour-treating activity, wherein “good” indicates that the chemical compound is likely to have paclitaxel-like tumour-treating activity and “bad” indicates that the tumour is not likely to have paclitaxel-like tumour-treating activity, and wherein the gene marker set is as defined in claim 1.

9. The method according to any one of claims 1 to 8, wherein
each gene in the gene expression profile has a gene expression value and a modified gene expression profile is obtained by multiplying the gene expression value by its marker-factor,
the standardized “good” and “bad” profiles are determined by computing standardized centroids for both “good” and “bad” classes using prediction analysis for microarrays method,
modified class centroids of the marker set are obtained by multiplying the standardized centroids for each class by the marker-factor, and
the modified gene expression profile of the sample is compared to each modified class centroid to determine the tumour is “good” or “bad”, wherein the class whose centroid is closest to the modified gene expression profile, in Pearson correlation distance, is predicted to be the class for the sample.

10. The method according to any one of claims 1 to 9, further comprising obtaining an output of the gene expression profile of the sample before comparing the gene expression profile to the standardized “good” and “bad” profiles of the marker set.

11. The method according to any one of claims 1 to 10, wherein the gene expression profile of the sample is determined by screening the sample against gene probes of the gene marker set using microarray analysis, individual gene screening, individual RNA screening, a diagnostic panel, a mini chip, a NanoString chip, a RNA-seq chip, a protein chip or an ELISA test.

12. The method according to any one of claims 1 to 10, wherein the gene expression profile of the sample is determined by screening the sample against a microarray on which gene probes of the marker set are printed.

13. Use of one or more of the gene marker sets as defined in claim 1 for predicting effectiveness of paclitaxel or a paclitaxel-like drug for treating a tumour.

14. The use according to claim 13, wherein all three of Sets 1, 2 and 3 or all three of Sets 4, 5 and 6 are used for the predicting.

15. The use according to claim 13 or 14, wherein the tumour is a breast tumour, ovarian tumour, lung tumour or prostate tumor.

16. A kit for predicting the effectiveness of paclitaxel or a paclitaxel-like drug for treating a tumour, the kit comprising gene probes for each of the genes in a gene marker set as defined in claim 1 along with instructions for obtaining a gene expression profile of a sample for the gene marker set.

17. The kit according to claim 16 comprising gene probes for all three of Sets 1, 2 and 3 or all three of Sets 4, 5 and 6.

18. The kit according to any one of claims 16 to 17, further comprising instructions for comparing the gene expression profile of the sample to standardized “good” and “bad” profiles of the marker set to determine whether the gene expression profile of the sample predicts that the tumour is treatable or untreatable by paclitaxel or a paclitaxel-like drug.
